(19) United States
(12) Patent Application Publication
(10) Pub. No.: US 2018/0207032 A1
(43) Pub. Date: Jul. 26, 2018

(54) VACUUM CONTROL FOR A VITRECTOMY PROBE

(71) Applicant: Novartis AG, Basel (CH)

(72) Inventors: Steven T. Charles, Memphis, TN (US); Brian William McDonell, Irvine, CA (US)

(21) Appl. No.: 15/866,828

(22) Filed: Jan. 10, 2018

Related U.S. Application Data
(60) Provisional application No. 62/450,672, filed on Jan. 26, 2017.

(51) Int. Cl. A61F 9/007

(52) U.S. Cl. CPC A61F 9/00763; A61F 2009/00874; A61F 9/00745

(57) ABSTRACT

Vitrectomy probes and methods related thereto are disclosed herein. A vitrectomy probe may include a hand piece comprising a housing, a venturi, a piloted proportional valve disposed in the housing, and aspiration tubing fluidly coupled to the piloted proportional valve. In some embodiments, the piloted proportional valve and/or venturi may be disposed in the surgical console. The vitrectomy probe may further include a cutting mechanism attached to a distal end of the hand piece, wherein the cutting mechanism may be fluidly coupled to the aspiration tubing.

### BACKGROUND

### PRIORITY CLAIM

This application claims the benefit of priority of U.S. Provisional Patent Application Ser. No. 62/450,672 titled “VACUUM CONTROL FOR A VITRECTOMY PROBE”, filed on Jan. 26, 2017, whose inventors are Steven T. Charles and Brian William McDonell, which is hereby incorporated by reference in its entirety as though fully and completely set forth herein.

Microsurgical procedures may frequently require precision cutting and/or removing various body tissues. For example, certain ophthalmic surgical procedures may require cutting and removing portions of the vitreous humor, a transparent jelly-like material that fills the posterior segment of the eye. The vitreous humor, or vitreous, is composed of numerous microscopic fibrils that are often attached to the retina. Therefore, cutting and removing the vitreous must be done with great care to avoid traction on the retina, the separation of the retina from the choroid, a retinal tear, or, in the worst case, cutting and removal of the retina itself. In particular, delicate operations such as mobile tissue management (e.g., cutting and removal of vitreous near a detached portion of the retina or a retinal tear), vitreous base dissection, and cutting and removal of membranes may be particularly difficult.

Vitrectomy probes may typically be inserted via an incision in the sclera in the pars plana. The surgeon may also insert other microsurgical instruments, such as a fiber optic endoilluminator, an infusion cannula, or an aspiration cannula during the posterior segment surgery. While performing the surgery, the surgeon may view the eye using a microscope. Vitrectomy probes may typically include an inner cutter needle and outer needle arranged coaxially with and movably disposed within the needle, and a port extending radially through the outer needle near the distal end thereof. Vitreous and/or membranes may be aspirated into the open port, and the cutter may be actuated, closing the port. Upon the closing of the port, cutting surfaces on both the inner cutter needle and outer needle may cooperate to cut the vitreous and/or membranes, and the cut tissue may then be aspirated away through the cutter. Additional vitrectomy probes may include laser cutters and ultrasonic cutters, among others. Tubing connecting a cutter with a console/cassette/vacuum valve(s) may cause a delay (e.g., speed of sound in fluid) and capacitance effects. The tubing may contract when a vacuum is applied, and may expand causing a residual vacuum when console/cassette vacuum commands decrease. The console/cassette vacuum commands may be controlled by a surgeon foot pedal.

### SUMMARY

In an exemplary aspect, the present disclosure is directed to a vitrectomy probe including a hand piece, a housing, a piloted proportional valve disposed in the housing, and aspiration tubing fluidly coupled to the piloted proportional valve. The vitrectomy probe may include a cutting mechanism attached to a distal end of the hand piece, and the cutting mechanism may be fluidly coupled to the aspiration tubing.

In another exemplary aspect, the present disclosure is directed to a vitrectomy probe including a hand piece that includes a housing, a venturi disposed in the housing, and aspiration tubing fluidly coupled to the venturi. The vitrectomy probe may include a supply line fluidly coupled to the venturi and a surgical console. The vitrectomy probe may include a cutting mechanism attached to a distal end of the hand piece, and the cutting mechanism may be fluidly coupled to the aspiration tubing.

In another exemplary aspect, the present disclosure is directed to a system that includes a vitrectomy probe. The vitrectomy probe may include a hand piece that includes a housing, a piloted proportional valve that may be disposed in the housing, and aspiration tubing that may be fluidly coupled to the piloted proportional valve. The vitrectomy probe may include a cutting mechanism attached to a distal end of the hand piece, and the cutting mechanism may be fluidly coupled to the aspiration tubing. The system may include a surgical console coupled to the vitrectomy probe.

In another exemplary aspect, the present disclosure is directed to a method for operating a vitrectomy probe. The method may include positioning a cutting mechanism extending from the vitrectomy probe in an eye, and the vitrectomy probe may include a hand piece that includes a housing, a piloted proportional valve that may be disposed in the housing, and aspiration tubing that may be fluidly coupled to the piloted proportional valve. The vitrectomy probe may include a cutting mechanism attached to a distal end of the hand piece, and the cutting mechanism may be fluidly coupled to the aspiration tubing. The method may further include cutting tissue within the eye with the cutting mechanism and aspirating material from the eye through the cutting mechanism and the hand piece.

The different aspects may include one or more of the following features. The piloted proportional valve may be controlled by an electrical signal or a pressure signal. The piloted proportional valve may include an inlet coupled to the aspiration tubing for receiving aspirated material from the cutting mechanism and an outlet for discharging the aspirated material. The outlet may be coupled to a vacuum source. The piloted proportional valve may be operable to control flow of aspirated material from the cutting mechanism through the hand piece. The cutting mechanism may be, for example, an axial mechanical cutter, a rotary mechanical cutter, an ultrasonic cutter, or a laser cutter. The hand piece may further include a venturi fluidly coupled to the piloted proportional valve and fluidly coupled to the aspiration tubing. The venturi may include an entry cone, an exit cone, and a flow constriction disposed between the entry cone and the exit cone. The venturi may be configured to create a pressure drop to draw aspirated material from the eye through the aspiration tubing. The venturi may include an inlet between the entry cone and the exit cone; the inlet being coupled to the aspiration tubing for receiving aspirated material from the cutting mechanism. The entry cone may be coupled to the piloted proportional valve for receiving a supply fluid from the piloted proportional valve, and the exit cone may discharge a mixture of the supply fluid and the aspirated material from the venturi. A supply line may be fluidly coupled to an inlet of the piloted proportional valve for receiving the supply fluid. The piloted proportional valve may include an exit for discharging the supply fluid to the venturi, and the piloted proportional valve may be operable to control flow of the supply fluid to the venturi.

The different aspects may include one or more of the following features. The piloted proportional valve may include an inlet coupled to the aspiration tubing for receiving aspirated material from the cutting mechanism and an outlet for discharging the aspirated material. The surgical console may include a vacuum source coupled to the outlet. The piloted proportional valve may be operable to control flow of aspirated material from the cutting mechanism through the hand piece. A line may couple the surgical console to the piloted proportional valve such that the line may be configured to provide an electrical signal or pressure signal from the surgical console to the piloted proportional valve. The cutting mechanism may be, for example, an axial mechanical cutter, a rotary mechanical cutter, an ultrasonic cutter, or a laser cutter. The hand piece may further include a venturi fluidly coupled to the piloted proportional valve and fluidly coupled to the aspiration tubing, and the venturi may include an entry cone, an exit cone, and a flow constriction disposed between the entry cone and the exit cone (the venturi being configured to create a pressure drop to draw aspirated material from the eye through the aspiration tubing). The venturi may include an inlet between the entry cone and the exit cone; the inlet being coupled to the aspiration tubing for receiving aspirated material from the cutting mechanism. The entry cone may be coupled to the piloted proportional valve for receiving a supply fluid from the piloted proportional valve, and the exit cone may discharge a mixture of the supply fluid and the aspirated material from the venturi to the surgical console. A supply line may be fluidly coupled to an inlet of the piloted proportional valve for receiving the supply fluid. The piloted proportional valve may include an exit for discharging the supply fluid to the venturi, and the piloted proportional valve may be operable to control flow of the supply fluid to the venturi. The surgical console may include a vacuum source, and a tubing may couple the vacuum source to the hand piece.

The different aspects may include one or more of the following features. The vitrectomy probe may include a piloted proportional valve disposed in the hand piece, and the piloted proportional valve may be fluidly coupled to the venturi and the surgical console.

The different aspects may include one or more of the following features. The material may pass through the piloted proportional valve to a surgical console. The surgical console may include a vacuum source that may provide vacuum pressure to the aspiration tubing. The hand piece may further include a venturi, and the material may pass through the aspiration tubing and the venturi.

It is to be understood that both the foregoing general description and the following drawings and detailed description are exemplary and explanatory in nature and are intended to provide an understanding of the present disclosure without limiting the scope of the present disclosure. It is also to be understood that the components described herein may be arranged in a different order or arranged in different locations in the system. For example, the piloted proportional valve and/or venturi may be located in the surgical console instead of the hand piece. Additional aspects, features, and advantages of the present disclosure will be apparent to one skilled in the art from the following.

### BRIEF DESCRIPTION OF THE DRAWINGS

For a more complete understanding of the present disclosure, reference is made to the following description taken in conjunction with the accompanying drawings in which:

FIG. 1 illustrates an example vitrectomy probe with a piloted proportional valve.

FIG. 2A illustrates an example vitrectomy probe with a piloted proportional valve and a venturi.

FIG. 2B illustrates an example vitrectomy probe with a venturi, and the vitrectomy probe is connected to a console containing the piloted proportional valve.

FIGS. 3A-3D illustrate an example cutting cycle for a vitrectomy probe.

FIG. 4 illustrates an example of a cutting mechanism being inserted into a posterior segment of an eye.

FIG. 5 illustrates a flowchart of a method for operating a vitrectomy probe.

### DETAILED DESCRIPTION

For the purposes of promoting an understanding of the principles of the present disclosure, reference will now be made to the implementations illustrated in the drawings and specific language will be used to describe them. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with reference to one or more implementations may be combined with the features, components, and/or steps described with reference to other implementations of the present disclosure. For simplicity, in some instances the same reference numbers are used throughout the drawings to refer to the same or like parts.

The present disclosure generally relates to a vitrectomy probe and associated methods of use. More particularly, embodiments may generally relate to vitrectomy probes that may include a venturi and/or a piloted proportional valve in the hand piece. In some embodiments, the piloted proportional valve and/or venturi may be located in the surgical console instead of the hand piece. A separate pneumatic pressure source line may drive the venturi. By inclusion of the venturi and/or piloted proportional valve in the hand piece, response time may be decreased. For example, a surgeon's response time may be no less than 400 milliseconds (“ms”), wherein it may be desired for the aspiration fluidic system to cause minimal additional delay. With a reduction in response time, safety may be improved. For example, there may be a decrease in the likelihood/number of retinal tears created by pulling on the vitreous and therefore procedures on the retina and/or movement of a detached retina toward a port of a cutting mechanism, may be improved.

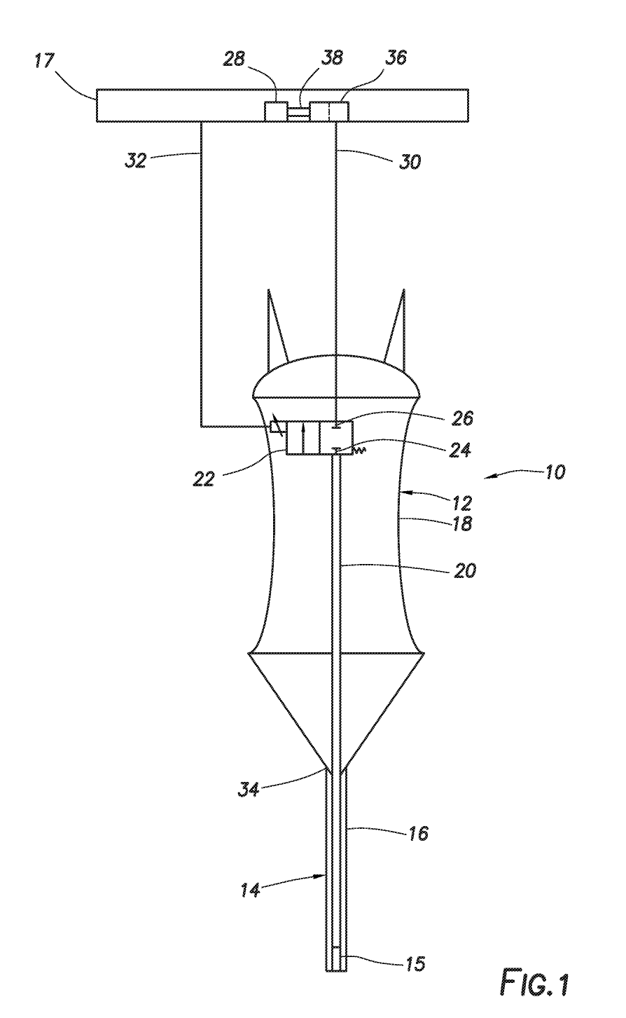

FIG. 1 illustrates an example of a vitrectomy probe 10. Vitrectomy probe 10 may comprise hand piece 12 and a cutting mechanism 14. Vitrectomy probe 10 may be coupled (e.g., fluidly and/or electrically) to surgical console 17.

The hand piece 12 may comprise a housing 18 containing an aspiration tubing 20 and a piloted proportional valve 22 disposed in the housing 18. The aspiration tubing 20 may be fluidly coupled to the cutting mechanism 14 for aspirating material, such as fluid and tissue, from the cutting mechanism 14. The aspiration tubing 20 may also be fluidly coupled to the piloted proportional valve 22. The piloted proportional valve 22 may be operable to control the flow of aspirated material from the cutting mechanism 14 through the hand piece 12. Proportional valves are generally valves for which the output value (e.g., pressure or flow) may be changed relative to the inlet value. The piloted proportional valve 22 may include any suitable type of proportional valve, including, but not limited to, poppet valves, ball valves, and spindle valves, among others. As illustrated, the piloted proportional valve 22 may include an inlet 24 and an outlet 26. The inlet 24 may be coupled to aspiration tubing 20 for receiving aspirated material from the cutting mechanism 14. The outlet 26 may discharge the aspirated material from piloted proportional valve 22. The outlet 26 may be coupled to surgical console 17, for example, by tube 30. The piloted proportional valve 22 may open or close based on an electrical signal or pressure signal (e.g., analog pressure due to a fluid) sent from the surgical console 17 via the line 32, thereby controlling aspiration of material, such as tissue and/or fluid, from the eye via the cutting mechanism 14. The piloted proportional valve 22 may change an output value (e.g., vacuum and/or flow) in proportion to an input value (e.g., pressure and/or flow) from the surgical console 17.

In some embodiments, the cutting mechanism 14 may comprise an inner needle 15 and an outer needle 16. In the illustrated embodiment, the inner needle 15 may be coaxially arranged in the outer needle 16. In some embodiments, the inner needle 15 and the outer needle 16 may both be tubular in shape with a hollow bore. In the illustrated embodiment, the cutting mechanism 14 may be attached to the hand piece 12 at its distal end 34. The cutting mechanism 14 may comprise any suitable cutter, such as, for example, a rotary mechanical cutter, an axial mechanical cutter (e.g., a pneumatically driven axial cutter), an ultrasonic cutter or a laser cutter.

With continued reference to FIG. 1, power may be supplied to the vitrectomy probe 10 via a power cable. The power cable may be coupled to the surgical console 17, and the surgical console 17 may be operable to adjust the power applied to the vitrectomy probe 10 based, for example, on an input to the surgical console 17 by a user, such as, for example, a surgeon. Input from a user to the surgical console 17 may be provided via an input device, such as, for example, a touch screen, button, slider, footswitch, and/or other input device. In some embodiments, the surgical console 17 may be coupled (e.g., fluidly coupled or electrically coupled) to the piloted proportional valve 22 via a line 32 (e.g., wire or tube). As illustrated, a vacuum source 36 may be disposed in surgical console 17. In some embodiments, the piloted proportional valve 22 may be fluidly coupled to the vacuum source 36 via the tube 30. In some embodiments, surgical console 17 may also include cassette 28 for receiving and storing aspirated material from the cutting mechanism 14 by way of the hand piece 12. The cassette 28 may be fluidly coupled to vacuum source 36 (e.g., aspiration pathway 33). The cassette 28 may be changed for each patient and may cooperate with surgical console 17 to provide fluid aspiration. Cassette 28 may be used for positive displacement aspiration, vacuum-based aspiration, or both. The cassette 28 may include an aspiration pathway 38 coupled to the vacuum source 36 and may allow surgical console 17 to selectively drive aspiration with vacuum source 36.

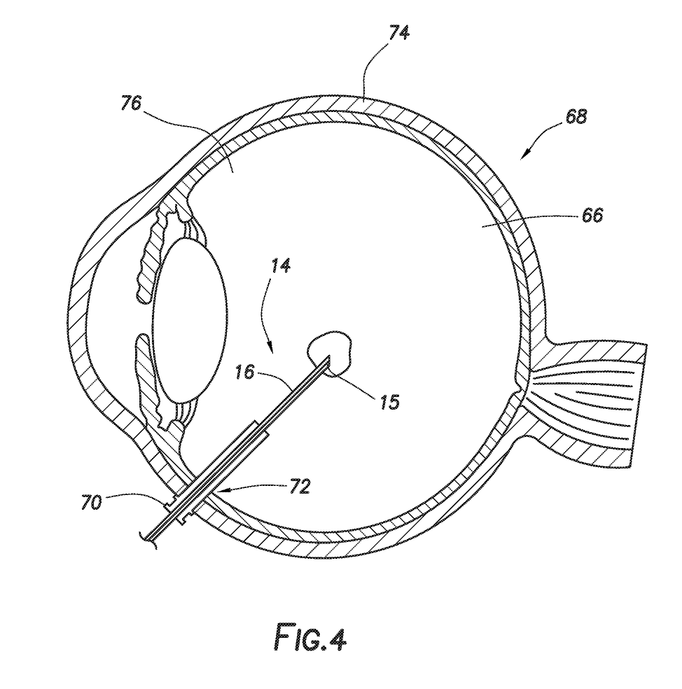

An example embodiment for operation of the vitrectomy probe 10 of FIG. 1 for aspiration of ophthalmic tissue during an ophthalmic surgical procedure will now be described. The cutting mechanism 14 may be operated to remove the ophthalmic tissue (e.g., vitreous humor 76 (interchangeably referred to as “vitreous”) on FIG. 4). Dissected tissue and/or fluid may be drawn into the cutting mechanism 14 and flow into the aspiration tubing 20 in the hand piece 12. The aspirated material may be received in piloted proportional valve 22 by way of the inlet 24. The aspirated material may be discharged from the piloted proportional valve 22 by way of the outlet 26. Aspirated material discharged from the piloted proportional valve 22 may be received by the cassette 28 in the surgical console 17 by way of tube 30. Vacuum source 36 may supply a vacuum pressure to the cutting mechanism 14. The piloted proportional valve 22 may control vacuum pressure as the inlet 24 and the outlet 26 open or close, thereby controlling the aspiration of material through the hand piece 12.

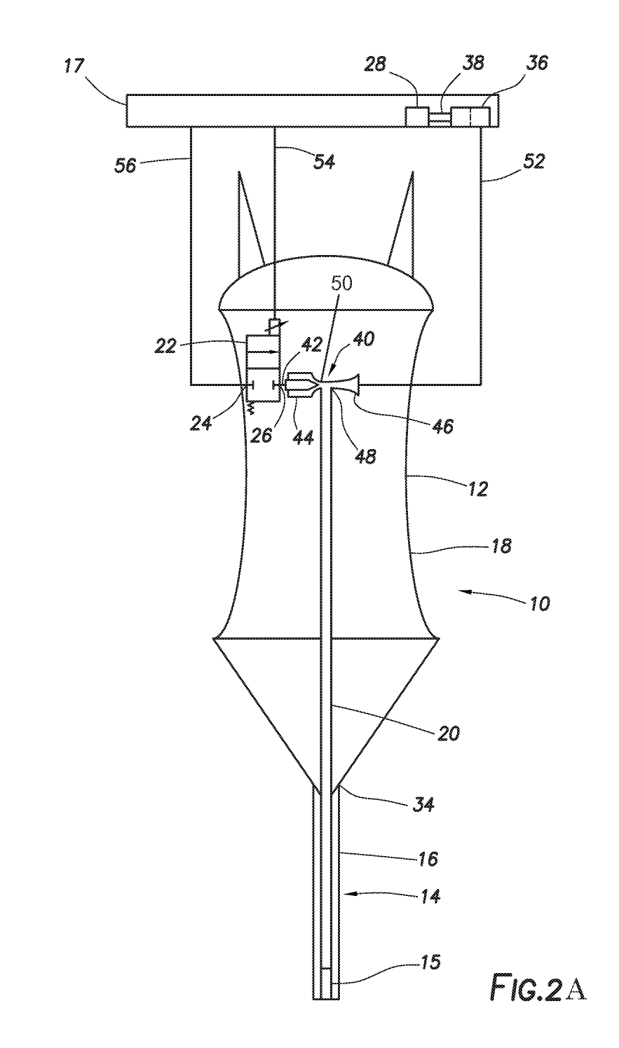

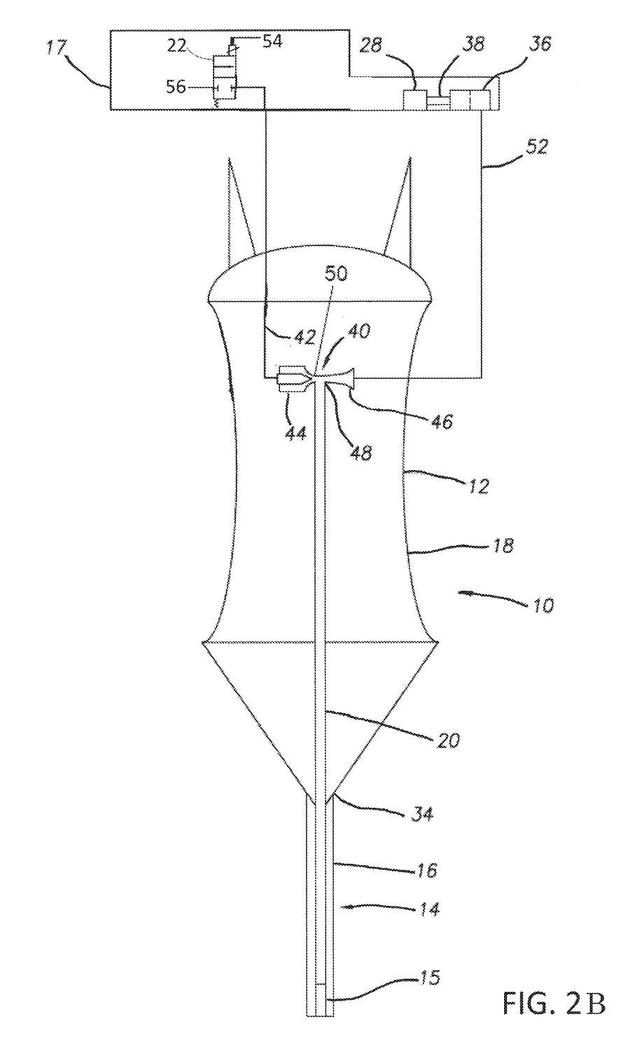

FIG. 2A illustrates another embodiment of the vitrectomy probe 10 that may further comprise a venturi 40 (e.g., a venturi tube). The vitrectomy probe 10 may comprise a hand piece 12 and a cutting mechanism 14. The hand piece 12 may comprise the venturi 40. The vitrectomy probe 10 may be coupled (e.g., fluidly and/or electrically) to a surgical console 17. While illustrated with a piloted proportional valve 22 in the hand piece 12, the venturi 40 may alternatively be controlled with a supply fluid sent directly from the surgical console 17.

The hand piece 12 may comprise a housing 18 containing an aspiration tubing 20, the venturi 40, and a piloted proportional valve 22, each being disposed, for example, in the housing 18. The aspiration tubing 20 may be fluidly coupled to the cutting mechanism 14 for aspirating material, such as fluid and tissue, from the cutting mechanism 14. The aspiration tubing 20 may also be fluidly coupled to the venturi 40, which may include an entry cone 44 and an exit cone 46. The entry cone 44 may be fluidly coupled to piloted proportional valve 22 via supply tube 56. A supply fluid may be delivered to the venturi 40 through the supply tube 56. The exit cone 46 may be fluidly coupled to tube 52. A mixture of the supply fluid and aspirated material may be discharged from the venturi 40 through the exit cone 46. The venturi 40 may further comprise an inlet 48 between the entry cone 44 and exit cone 46. The aspiration tubing 20 may be fluidly coupled to the inlet 48 for delivery of the aspirated material to the venturi 40. The venturi 40 may comprise a flow restriction 50 between the entry cone 44 and the exit cone 46. By way of example, the venturi 40 may have an hourglass shape that forms the flow restriction 50. The flow restriction 50 may be configured to vary flow characteristics of the supply fluid (e.g., liquid and/or gas) traveling through the venturi 40. As the supply fluid velocity in the venturi 40 is increased (e.g., via flow restriction 50), there may be a consequential drop in pressure, which may be referred to as the “Venturi Principle” or “Venturi Effect”. The venturi 40 may use this pressure drop (e.g., a pressure drop may cause a suction or vacuum) to draw aspirated material from the cutting mechanism 14 and into the inlet 48.

In some embodiments, the piloted proportional valve 22 may be operable to control flow of the supply fluid to the venturi 40. The piloted proportional valve 22 may include inlet 24 and outlet 26 which may open or close based on an electrical signal or pressure signal (e.g., analog pressure due to a fluid) sent from the surgical console 17 via the line 54, thereby controlling a flow of the supply fluid in supply tube 56 to the entry cone 44. The inlet 24 may be coupled to supply tube 56 for receiving a supply fluid (e.g., saline or sterile water), for example, from the surgical console 17. The outlet 26 may discharge the supply fluid from the piloted proportional valve 22. Supply tube 56 may couple the piloted proportional valve 22 to the venturi 40 to provide the supply fluid to the venturi 40 via the entry cone 44.

In some embodiments, the cutting mechanism 14 may comprise an inner needle 15 and an outer needle 16. In the illustrated embodiment, the inner needle 15 may be coaxially arranged in the outer needle 16. In some embodiments, the inner needle 15 and the outer needle 16 may both be tubular in shape with a hollow bore. In the illustrated embodiment, the cutting mechanism 14 may be attached to the hand piece 12 at its distal end 34. The cutting mechanism 14 may comprise any suitable cutter, such as, for example, a rotary mechanical cutter, an axial mechanical cutter (e.g., a pneumatically driven axial cutter), an ultrasonic cutter or a laser cutter.

With continued reference to FIG. 2A, power may be supplied to the vitrectomy probe 10 via a power cable. The power cable may be coupled to the surgical console 17, and the surgical console 17 may be operable to adjust the power applied to the vitrectomy probe 10 based, for example, on an input to the surgical console by a user, such as, for example, a surgeon. Input from a user to the surgical console 17 may be provided via an input device, such as, for example, a surgeon controlled proportional foot pedal. The surgical console 17 may be coupled (e.g., fluidly coupled or electrically coupled) to the piloted proportional valve 22 via a line 54 (e.g., wire or tube). The exit cone 46 may be fluidly coupled to a vacuum source 36 via a tube 52 (e.g., exhaust tube). The vacuum source 36 may be disposed in the surgical console 17. Surgical console 17 may also include cassette 28 for receiving and storing aspirated fluid and/or tissue. The cassette 28 may be fluidly coupled to vacuum source 36 (e.g., aspiration pathway 38). The cassette 28 may be changed for each patient and may cooperate with surgical console 17 to provide fluid aspiration. Cassette 28 may be used for positive displacement aspiration, vacuum-based aspiration, or both. The cassette 28 may include an aspiration pathway 38 coupled to the vacuum source 36 and may allow surgical console 17 to selectively drive aspiration with vacuum source 36.

An example embodiment for operation of the vitrectomy probe 10 of FIG. 2A for aspiration of ophthalmic tissue during an ophthalmic surgical procedure will now be described. The cutting mechanism 14 may be operated to remove the ophthalmic tissue (e.g., vitreous humor 76 (interchangeably referred to as “vitreous”) on FIG. 4). Dissected tissue and/or fluid may be drawn into the cutting mechanism 14 and flow into the aspiration tubing 20 in the hand piece 12. The aspirated material may be received in the venturi 40 by way of the inlet 48. In addition, the piloted proportional valve 22 may be operated to control an amount of the supply fluid to the entry cone 44. The supply fluid travels through the venturi 40 exiting by way of the exit cone 46. As the supply fluid velocity in the venturi 40 is increased (e.g., via flow restriction 50), there may be a consequential drop in pressure. The venturi 40 may use this pressure drop (e.g., a pressure drop may cause a suction or vacuum) to draw the aspirated material from the cutting mechanism and into the venturi 40 by way of the inlet 48. The aspirated material may mix with the supply fluid in the venturi 40 and be discharged from the venturi 40 by way of the exit cone 46. Aspirated material discharged from the venturi 40 may be received by the cassette 28 in the surgical console 17 by way of the tube 52. In some embodiments, the vacuum source 36 may supply a vacuum pressure to the cutting mechanism 14.

FIG. 2B illustrates a similar embodiment as FIG. 2A except the piloted proportional valve 22 is located in the surgical console 17 and the venturi drive line 42 extends from the piloted proportional valve 22 in the console 17 to the venturi 40 in the handpiece (e.g., through a flexible tube). As shown in FIG. 2B, the supply tube 56 and the signal line 54 (receiving the electrical signal or pressure signal) may both be internal to the console 17 and connected to the piloted proportional valve 22. In some embodiments, the venturi valve may also be located in the control console (in which case, aspiration tubing 20 may extend from the hand piece to the console). Other configurations are also contemplated.

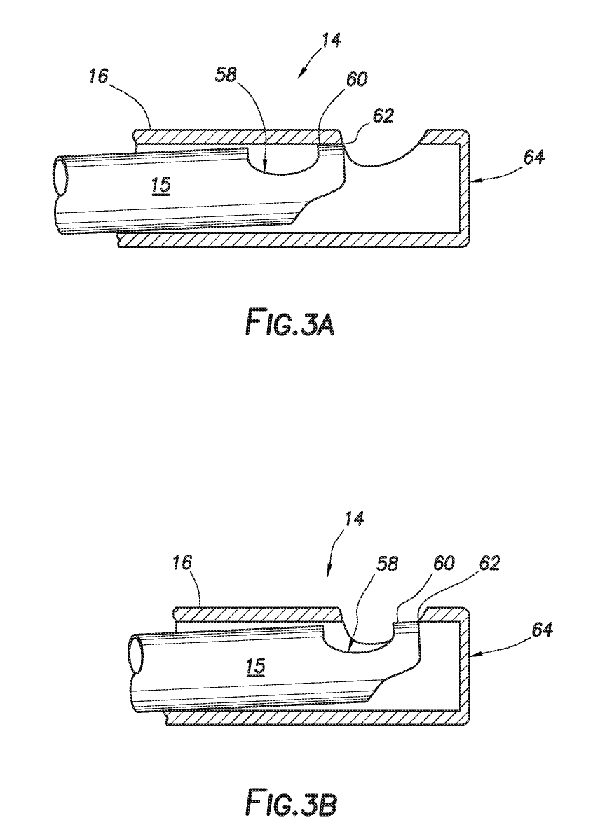

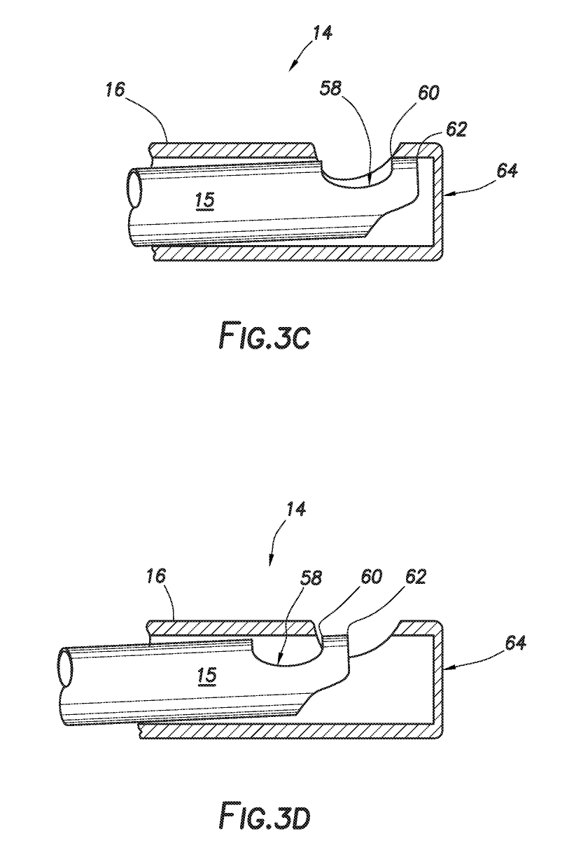

FIGS. 3A-3D illustrates a detailed view of an example of the cutting mechanism 14 in accordance with example embodiments of the present disclosure. Although an axial mechanical cutter is illustrated in FIGS. 3A-3D, other suitable cutters, such as, for example, a rotary mechanical cutter, oscillating rotary cutter, an ultrasonic cutter or a laser cutter may be used. In some embodiments, the inner needle 15 may be in the form of a hollow cylinder, but other configurations of the cutter 14 may also be suitable. As illustrated, a port 58 may be formed in the inner needle 15 that may receive various materials, such as tissue or fluid, during operation. In some embodiments, the tissue may be ophthalmic tissue, such as vitreous and/or membrane. The port 58 may be of a polygonal (e.g., rectangular) or other suitable shape. The inner needle 15 may be in the form of a single blade configuration or a dual blade or dual port configuration including two cutting edges, for example, proximal cutting edge 60 and distal cutting edge 62. The proximal cutting edge 60 may be formed at a distal side of the port 58. The distal cutting edge 62 may be formed at a distal side of the inner needle 15. When moving, the proximal cutting edge 60 and the distal cutting edge 62 may cut material, such as tissue. For example, the proximal cutting edge 60 and distal cutting edge 62 may cooperate with cutting edges on the outer needle 16 to cut the material. The inner needle 15 may be made of any suitable material, including surgical stainless steel. The inner needle 15 may be of any suitable dimensions, including, but not limited to, a length of about 1 inch to about 2 inches. Additionally, in some embodiments, the inner needle 15 may have a size that ranges from about 23 gauge to about 27 gauge. One of ordinary skill in the art, with the benefit of this disclosure, should be able to select the dimensions and operating parameters for use of the inner needle 15 in a particular application.

With reference now to FIGS. 3A-3D, an example embodiment for operation of the cutting mechanism 14 in the form of an axial mechanical cutter will now be described. FIG. 3A represents a stage in the cutting cycle where the inner needle 15 is in the open position. In this open position, vacuum pressure (e.g., from vacuum source 36 on FIGS. 1 and 2) in the inner needle 15 may pull or aspirate tissue into the outer needle 16. As shown in FIG. 3B, the inner needle 15 may travel distally towards distal end 64 of the outer needle 16. As the inner needle 15 moves forward, the distal cutting edge 62 may cut tissue that has entered the outer needle 16. As illustrated in FIG. 3C, the inner needle 15 may continue to move distally further into the outer needle 16. While not shown, the inner needle 15 may move until the distal cutting edge 62 becomes substantially flush with a distal end 64 of the outer needle 16. In this position, vacuum pressure (e.g., from vacuum source 36 on FIGS. 1 and 2) in the inner needle 15 may pull or aspirate tissue into the inner needle 15 by way of port 58. After the inner needle 15 moves distally towards the distal end 64, the inner needle 15 may move proximally (backwards, i.e., away from distal end 64), as illustrated in FIG. 3D. As inner needle 15 moves proximally, proximal cutting edge 60 may cut tissue. Aspirated material, including fluid and/or severed tissue may be drawn through the cutting mechanism to the hand piece 12 (e.g., shown on FIGS. 1 and 2).

Referring now to FIG. 4, use of cutting mechanism 14 in an ophthalmic surgical procedure will now be described in accordance with an example embodiment. As illustrated in FIG. 4, during an ophthalmic surgical procedure, such as a retinal surgical procedure, the inner needle 15 and the outer needle 16 may be inserted into the posterior segment 66 of the eye 68. For example, the inner needle 15 and the outer needle 16 may be inserted through a cannula 70 disposed in an incision 72. The incision 72 may be made through the sclera 74 of the eye 68. Once positioned, the cutting mechanism 14 may be operable to remove and aspirate ophthalmic tissue, such as vitreous and/or membrane. For example, during a retinal surgical procedure, the outer needle 16 with inner needle 15 disposed therein, may be inserted into the posterior segment 66 of the eye 68. The cutting mechanism 14 may be operated to remove the ophthalmic tissue, which may include vitreous humor 76 (interchangeably referred to as “vitreous”), a jelly-like substance that occupies the volume defined by the posterior segment 66, as the inner needle 15 moves back and forth within outer needle 16. The inner needle 15 may also be used to remove membranes covering the retina or other tissues. Dissected tissue and/or fluid from the eye 68 may be removed via the outer needle 16, as mentioned above (e.g., shown in FIGS. 3A-3D). Although an axial mechanical cutter is illustrated in FIG. 4, other suitable cutters, such as, for example, a rotary mechanical cutter, an ultrasonic cutter or a laser cutter may be used.

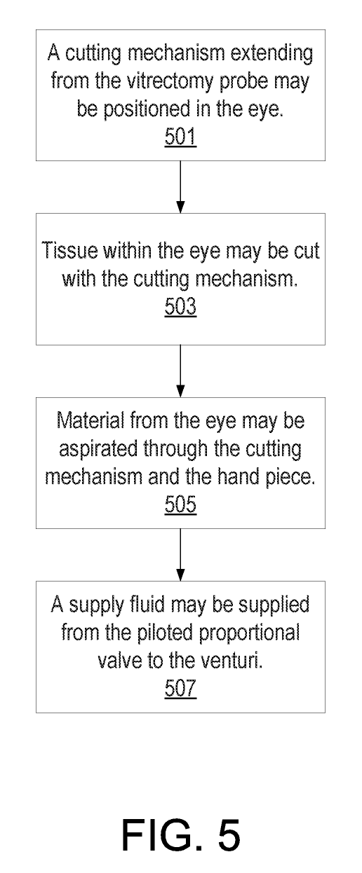

FIG. 5 illustrates a flowchart of a method for operating a vitrectomy probe. The elements provided in the flowchart are illustrative only. Various provided elements may be omitted, additional elements may be added, and/or various elements may be performed in a different order than provided below.

At 501, a cutting mechanism extending from the vitrectomy probe may be positioned in an eye. The vitrectomy probe may include a hand piece comprising a housing, a piloted proportional valve disposed in the housing, and aspiration tubing fluidly coupled to the piloted proportional valve. The cutting mechanism may be attached to a distal end of the hand piece, and the cutting mechanism may be fluidly coupled to the aspiration tubing.

At 503, tissue within the eye may be cut with the cutting mechanism.

At 505, material from the eye may be aspirated through the cutting mechanism and the hand piece. In some embodiments, the material may pass through the piloted proportional valve to a surgical console that has a vacuum source providing vacuum pressure to the aspiration tubing. In some embodiments, the hand piece may further include a venturi and the material may pass through the aspiration tubing and the venturi.

At 507, a supply fluid may be supplied from the piloted proportional valve to the venturi.

It is believed that the operation and construction of the present disclosure will be apparent from the foregoing description. While the apparatus and methods shown or described above have been characterized as being preferred, various changes and modifications may be made therein without departing from the spirit and scope of the disclosure as defined in the following claims.

What is claimed is:

1. A vitrectomy probe comprising:
a hand piece comprising a housing, a piloted proportional valve disposed in the housing, and aspiration tubing fluidly coupled to the piloted proportional valve; and
a cutting mechanism attached to a distal end of the hand piece, wherein the cutting mechanism is fluidly coupled to the aspiration tubing.

2. The vitrectomy probe of claim 1, wherein the piloted proportional valve is controlled by an electrical signal or a pressure signal.

3. The vitrectomy probe of claim 1, wherein the piloted proportional valve comprises an inlet coupled to the aspiration tubing for receiving aspirated material from the cutting mechanism and an outlet for discharging the aspirated material, the outlet being coupled to a vacuum source.

4. The vitrectomy probe of claim 1, wherein the piloted proportional valve is operable to control flow of aspirated material from the cutting mechanism through the hand piece.

5. The vitrectomy probe of claim 1, wherein the cutting mechanism is an axial mechanical cutter, a rotary mechanical cutter, an ultrasonic cutter, or a laser cutter.

6. The vitrectomy probe of claim 1, wherein the hand piece further comprises a venturi fluidly coupled to the piloted proportional valve and fluidly coupled to the aspiration tubing.

7. The vitrectomy probe of claim 6, wherein the venturi comprises an entry cone, an exit cone, and a flow constriction disposed between the entry cone and the exit cone, the venturi being configured to create a pressure drop to draw aspirated material from an eye through the aspiration tubing.

8. The vitrectomy probe of claim 7, wherein the venturi comprises an inlet between the entry cone and the exit cone, the inlet being coupled to the aspiration tubing for receiving aspirated material from the cutting mechanism.

9. The vitrectomy probe of claim 8, wherein the entry cone is coupled to the piloted proportional valve for receiving a supply fluid from the piloted proportional valve, wherein the exit cone discharges a mixture of the supply fluid and the aspirated material from the venturi.

10. The vitrectomy probe of claim 7, wherein a supply line is fluidly coupled to an inlet of the piloted proportional valve for receiving a supply fluid, the piloted proportional valve comprising an exit for discharging the supply fluid to the venturi, wherein the piloted proportional valve is operable to control flow of the supply fluid to the venturi.

11. A vitrectomy probe comprising:
a hand piece comprising a housing, a venturi disposed in the housing, and aspiration tubing fluidly coupled to the venturi;
a venturi drive line coupled to the venturi on one end and to a piloted proportional valve disposed in a surgical console on the other end; and
a cutting mechanism attached to a distal end of the hand piece, wherein the cutting mechanism is fluidly coupled to the aspiration tubing.

12. The vitrectomy probe of claim 11, further comprising an exhaust tube coupled between the surgical console and an output of the venturi.

13. A system comprising:
a vitrectomy probe comprising:
a hand piece comprising a housing, a venturi disposed in the housing, and aspiration tubing fluidly coupled to the venturi; and
a cutting mechanism attached to a distal end of the hand piece, wherein the cutting mechanism is fluidly coupled to the aspiration tubing; and
a surgical console coupled to the vitrectomy probe.

14. The system of claim 13, further comprising a piloted proportional valve coupled to the venturi, wherein the piloted proportional valve is disposed in the handpiece or the surgical console.

15. The system of claim 13, wherein the venturi comprises an inlet coupled to the aspiration tubing for receiving aspirated material from the cutting mechanism and an outlet for discharging the aspirated material, the surgical console comprising a vacuum source coupled to the outlet.

16. The system of claim 13, wherein the venturi is operable to control flow of aspirated material from the cutting mechanism through the hand piece, wherein the system further comprises a venturi drive line between the piloted proportional valve and the venturi.

17. The system of claim 14, wherein the venturi comprises an entry cone, an exit cone, and a flow constriction disposed between the entry cone and the exit cone, the venturi being configured to create a pressure drop to draw aspirated material from an eye through the aspiration tubing.

18. The system of claim 17, wherein the venturi comprises an inlet between the entry cone and the exit cone, the inlet being coupled to the aspiration tubing for receiving aspirated material from the cutting mechanism, wherein the entry cone is coupled to the piloted proportional valve for receiving a supply fluid from the piloted proportional valve, wherein the exit cone discharges a mixture of the supply fluid and the aspirated material from the venturi to the surgical console.

19. The system of claim 17, wherein a supply line is fluidly coupled to an inlet of the piloted proportional valve for receiving a supply fluid, the piloted proportional valve comprising an exit for discharging the supply fluid to the venturi, wherein the piloted proportional valve is operable to control flow of the supply fluid to the venturi.

20. The system of claim 13, wherein the surgical console comprises a vacuum source, wherein a tubing couples the vacuum source to the hand piece.
